(19) United States
(12) Patent Application Publication
(10) Pub. No.: US 2015/0293070 A1
(43) Pub. Date: Oct. 15, 2015

(54) ON-SITE KIT FOR ANALYSIS OF DISINFECTANT BYPRODUCTS SPECIES AND AMOUNTS THEREOF IN DRINKING WATER SUPPLIES

(71) Applicants: Gary L. Emmert, Collierville, TN (US); Paul S. Simone, JR., Collierville, TN (US); Yin Yee Choo, Memphis, TN (US); Meggan Larson, Brooklyn Park, MN (US); Patricia L. Ranaivo, Cordova, TN (US)

(72) Inventors: Gary L. Emmert, Collierville, TN (US); Paul S. Simone, JR., Collierville, TN (US); Yin Yee Choo, Memphis, TN (US); Meggan Larson, Brooklyn Park, MN (US); Patricia L. Ranaivo, Cordova, TN (US)

(21) Appl. No.: 14/126,809

(22) PCT Filed: Jun. 15, 2012
(86) PCT No.: PCT/US12/42766

§ 371 Date: Sep. 18, 2014

Related U.S. Application Data
(60) Provisional application No. 61/497,464, filed on Jun. 15, 2011.

(51) Int. Cl. G01N 33/18; G01N 21/64

(52) U.S. Cl. CPC G01N 33/182; G01N 21/6428

(57) ABSTRACT

The present invention relates to the provision of a portable kit that permits reliable and quantifiable analysis of trihaloamethanes and haloacetic acids within drinking water samples utilizing a hand-held (portable) fluorescing detection instrument for simultaneous measurements of such species. With the necessity to chlorinate drinking water to remove harmful bacteria and other potential toxins, trihalomethane and haloacetic acid byproducts are generated during such a disinfecting procedure that may harm humans after consumption as well due to highly suspect carcinogenicity. A reliable manner of measuring such drinking water supplies for such disinfectant byproduct levels is highly desirable, particularly through the utilization of a relatively inexpensive analytical instrument for such a purpose. The inventive kit-based analytical method of the invention has been found to be nearly as reliable as source measuring methods for the same purpose, providing a relatively quick measuring method that may be undertaken at any drinking water source location.

### REFERENCE TO CORRELATED PATENT APPLICATIONS

This application is the national stage filing of Patent Cooperation Treaty Patent Application PCT/US12/42766, filed on Jun. 15, 2012, currently pending, which claimed priority from U.S. Provisional Patent Application 61/497,464, expired, filed on Jun. 15, 2011. The specification and drawings of such prior applications are hereby entirely incorporated within.

### FIELD OF THE INVENTION

The present invention relates to the provision of a portable kit that permits reliable and quantifiable analysis of trihaloamethanes and haloacetic acids within drinking water samples utilizing a hand-held fluorimeter for simultaneous measurements of such species. With the necessity to chlorinate drinking water to remove harmful bacteria and other potential toxins, trihalomethane and haloacetic acid byproducts are generated during such a disinfecting procedure that may harm humans after consumption as well due to highly suspect carcinogenicity. A reliable manner of measuring such drinking water supplies for such disinfectant byproduct levels is highly desirable, particularly through the utilization of a relatively inexpensive analytical instrument for such a purpose. The inventive kit-based analytical method of the invention has been found to be nearly as reliable as source measuring methods for the same purpose, providing a relatively quick measuring method that may be undertaken at any drinking water source location.

### BACKGROUND OF THE INVENTION

Drinking water has been, and continues to be, heavily treated for bacteria and other microscopic organisms that may cause infection in humans and other animals subsequent to consumption. In order to disinfect water supplies, halogenated materials have been introduced therein that have proven more than adequate for such a purpose. Unfortunately, although such halogenated compounds (chlorinated and chloraminated types, primarily) exhibit excellent disinfection capabilities, when present within aqueous environments at certain pH levels these halogenated compounds may generate byproducts that may themselves create health concerns. The United States Environmental Protection Agency (USEPA) in fact currently regulates four types of trihalomethanes (THM4) and five specific types of haloacetic acids (HAM) within drinking water. These THM4 are chloroform, bromoform, dibromochloromethane, and bromodichloromethane, and these HAA5 are monochloroacetic acid, dichloroacetic acid, trichloroacetic acid, monobromoacetic acid, and dibromoacetic acid. Removal of such compounds from drinking water is not possible as for typical chlorinated disinfecting compounds, at least not at the same reliability level as for the disinfecting agents (the brominated species listed above may occur as the result of certain chlorinated acids and/or ions reacting with brominated compounds present within the drinking water prior to disinfection or hypobromous acid). Thus, residual amounts may remain within treated water supplies that may require further removal processes to be undertaken. Of course, if the level of contamination is sufficiently low, initiation of such potentially expensive removal steps would be unwise from an economic perspective.

The USEPA currently has set a maximum contaminant level for these THM4 in drinking water at 0.080 mg/L and for these HAM in drinking water at 0.060 mg/L (four other haloacetic acids are currently not regulated by the USEPA, bromochloroacetic acid, bromodichloroacetic acid, dibromochloroacetic acid, and tribromoacetic acid; including these, the total haloacetic acid group is known as HAA9). It is thus important to reliably analyze and measure the total amount of such contaminants in order to determine if removal if necessary. The USEPA has instituted its own testing methods for such a purpose. Four such methods are currently in practice to measure HAM levels: USEPA 552 and 552.2, which involve the liquid-liquid extraction of haloacetic acids from water sources into methyl-t-butyl ether, followed by derivatization with acidic methanol to form the corresponding haloacetic acid methyl esters. Analysis by gas chromatography-electron capture detection provides reliable measurements of the haloacetic acid amounts present within the subject water supply. The USEPA 552.1 test protocol employs ion-exchange liquid solid extraction, subsequent derivatization into methyl esters, and similar gas chromatography-electron capture detection. The other, USEPA 552.3, is a derivative of the first with optimizations of acidic methanol neutralization procedures for improvement in recoveries for brominated trihalogenated haloacetic acid species. However, these general processes have been found to have numerous drawbacks. For instance, injection port temperature can affect debromination of certain haloacetic acid species (particularly tribrominated types) that may lead to underrepresentation of the amount of such contaminants present within the tested water source. Likewise the water content of the methyl-t-butyl ether extract may decarboxylate the haloacetic acids, again leading to an under-reporting of the actual amounts present within the test sample. Furthermore, the involved processing needed to actually undergo such analysis makes an on-line protocol rather difficult to implement, particularly when hourly sampling is necessary. Other derivatization methods have been either followed or suggested for gas chromatography analyses of drinking water sources as well, including utilizing diazomethane, acidic ethanol, and aniline. Such reactant-based measurements, however, all suffer the same time and labor-intensive problems as with the two USEPA test procedures noted above. As such, on-line analysis through these protocols are difficult, expensive, and labor intensive to implement and run.

Recently, other measurement protocols were developed utilizing capillary membrane separation techniques to sequester haloacetic acids from trihalomethanes, subsequently reacting such species with fluorescing compounds, and then detecting the fluorescence signatures of both reactants with large-scale detectors. Such prior methods and devices have been modified to permit analysis at remote locations in an on-line system, albeit with rather expensive and cumbersome detection instruments for such a purpose.

As such, there is now a drive to provide portable detection methods and devices that can be performed and operated by a person at any location and that allows for reliable measurements for water utilities and other water suppliers to undertake a USEPA compliance program through such a low-tech, economical manner. Such a desirable portable procedure has been difficult to achieve, however, particularly as it pertains to the determination of not only the total amount of THM4 and HAA9 within water supplies, but also the amount of each species of THM4 and HAA9 groups present within the tested water source. Prior testing protocols utilized a variety of analytical procedures, including high performance liquid chromatography, electrospray ionization-mass spectrometry, ultraviolet absorbance, inductively coupled plasma-mass spectrometry and electrospray ionization-mass spectrometry coupled with ion chromatography, as well as ion chromatography with membrane-suppressed conductivity detection or ultraviolet absorbance detection, for such a purpose. Such methods require stationary instruments, specific reaction conditions and environments, and/or other specific limitations in terms of location, at least, to effectuate a reliable measurement reading for such species. As well, the detection levels for the trihalomethane and haloacetic acid compounds present in drinking water samples may be too low for these prior protocols to function properly, at least for, again, reliable analyses on the level of USEPA requirements.

Another methodology that has proven effective is post-column reaction-ion chromatography. This has shown promise, but only in terms of quantifying bromate ion concentrations in drinking water samples at a single μg/L level. This dual selectivity form (separation by ion chromatography column as well as the selective reaction with the post-column reagent with the analyte) offers an advantageous test method over the others noted above, except for the presence of more common anions, specifically chloride, at much higher concentrations within the sampled drinking water supply (mg/L instead of μg/L). It was then undertaken to combine the separation capabilities of ion chromatography with the reaction of the haloacetic acid species with nicotinamide, followed by fluorescence detection to measure the individual and total HAAS concentrations in drinking water at the single μg/L level. The problem with such a protocol, unfortunately, was that bromochloroacetic acid interfered with dichloro- and dibromo-acetic acid quantifications. Despite this problematic limitation, it was determined that fluorescence detection provided a much-improved detection protocol in comparison with ultraviolet and mass spectrometry possibilities. Thus, although such a fluorescence method of detection, coupled with the post-column reaction (again with nicotinamide reagent) and ion chromatography, exhibited the best results in terms of an on-line test method for HAA5 drinking water contaminant measurement levels, there remained a definite need for improvements in total trihalomethane and haloacetic acid measurements and identifications within such test samples. To date, however, there has not been an analytical test protocol that has permitted implementation of such a system within an on-line, real-time monitoring procedure with an acceptable degree of reliability. A portable system that provides such versatility and reliability has simply not been forthcoming within the pertinent art.

### ADVANTAGES AND SUMMARY OF THE INVENTION

Accordingly, it is an advantage of the present invention to provide a reliable portable drinking water analytical protocol for determining the total measurements for both the four different trihalomethanes and nine different haloacetic acids that are commonly present as disinfection byproducts within such water sources. It is an additional advantage of the invention to provide reliability similar to that exhibited by USEPA 552 test method series described above, but through the utilization of a portable fluorescence monitoring device that has been properly calibrated and with measurements properly attenuated in view of difficulties inherent with such a protocol.

Accordingly, the instant invention encompasses a portable kit for analyzing drinking water samples comprising:
a) a capillary membrane sampling module comprising:

a capillary membrane sampling device, a 2-channel pump, and at least two volumetric flasks;
b) a nicotinamide fluorescence chemistry module comprising:

reagent supplies of nicotinamide and a base, and at least two vials for mixing said nicotinamide reagent and said base reagent with at least two different water stream samples;
c) a fluorescence detector module comprising:

a hand-held fluorescence detector and at least two cuvettes that may be introduced within said fluorescence detector; and
d) an accessories module comprising:

a covered water bath that may be heated to at least 80° C. and an ice bath, wherein both said baths can hold both volumetric flasks within said capillary membrane sampling module simultaneously, and a thermometer to monitor the temperature of both baths;

wherein all of said modules are simultaneously portable and carried within a single properly configured and compartmented case.

As well, the invention encompasses a method for analyzing drinking water samples through the utilization of a portable analytical device, comprising the steps of:

a) providing at least one stream of drinking water that has been disinfected with chlorinated or chloraminated materials;

b) providing a kit including four modules for analysis of said stream of drinking water in step “a”, said modules including i) a capillary membrane sampling module, ii) a nicotinamide fluorescence chemistry module, iii) a fluorescence detector module, and iv) an accessories module, wherein said modules may be properly operated by a single person throughout each procedure, and wherein said capillary membrane sampling module includes a capillary membrane sampling component including a tube-within-a-tube construction to allow two different streams of liquid to pass through said component simultaneously;

c) introducing said at least one drinking water stream into said capillary membrane sampling component through one tube and a reagent water stream through the other tube within said component such that device such that all volatile trihalomethane compounds present within said drinking water stream separates from said stream within said capillary membrane sampling device into a stream of reagent water, and wherein any haloacetic acid compounds will remain within said at least one stream of drinking water;

d) transporting both of said trihalomethane-containing stream of reagent water and said drinking water haloacetic acid-containing stream to separate reservoirs, wherein samples from each reservoir are removed therefrom and kept separate;

e) mixing said separate samples with a nicontinamide fluorescing compound within the nicotinamide fluorescing chemistry module;

f) optionally heating said nicotinamide-mixed separate samples to a temperature to effectuate reaction therewith between said separated trihalomethane compounds and said haloacetic acid compounds within different mixing vessels;

g) optionally cooling each resultant fluoresced separate sample thereof to a temperature that permits handling and introduction within a cuvette for placement within a fluorimeter, wherein said heating and cooling steps are undertaken within the accessories module;

h) introducing said resultant separate samples into separate cuvettes;

i) placing said separate cuvettes into a portable fluorescing detection instrument within the fluorescing detector module; and

j) operating said fluorescing detection instrument to determine the amount of total trihalomethane and haloacetic acid species within each resultant separate sample through fluorescence detection (and optionally measuring the amount of each different THM and HAA present within such separate samples).

Generally speaking, the inventive kit and method uses a capillary membrane sampling (CMS) device to separate the trihalomethanes (THMs) from the haloacetic acids (HAAs) present within disinfected drinking water, followed by reaction of the separated THMs and HAAs with nicotinamide (NCA) and base (typically sodium hydroxide), followed by detection of the fluorescence produced by the reaction products (excitation wavelength 360 nm region; emission wavelength 450 nm region). Although such a method has been followed in a broad sense, the invention specifically requires the utilization of a hand-held fluorimeter to perform the fluorescence detection steps which then lend themselves to measuring the amounts of THMs and HAAs within the drinking water samples themselves. Such a modification from previous methods may not seem significant on its face; however, the capability of portable fluorescing detection instruments (such as, for instance, a fluorimeter) in terms of reliability, particularly as compared with a chromatographic or other type or large-scale device, has proven suspect in the past. In this situation, various calibrations and modifications, not to mention calculation changes, have proven necessary to provide the degree of reliability required by and at least on par with USEPA guidelines for such test protocols. As such, there is no reason that the ordinarily skilled artisan would view the fluorimeter (or other portable fluorescing detection instrument) of the present invention as a proper substitute for previous analytical devices. To the contrary, it would appear, at first glance, as a step in the wrong direction in terms of reliable measurements and thus trustworthiness. Unexpectedly, then, the actions undertaken in developing this reliable kit and method have proven inordinately important to permitting the utilization of such a hand-held fluorescence detector in a remote location setting, all to provide proper measurements within very low detection limits of potentially harmful disinfectant by-products within drinking water supplies. Through this rather rudimentary, yet effective protocol, an economical and reliable system for drinking water treatment may be implemented.

As alluded to above, the CMS component of the capillary membrane sampling module includes a “tube-within-a-tube” configuration. A drinking water sample flows through the outer tube (made from Tefzel, for example), while reagent water flows through the inner tube (such as, for example, silicone rubber membrane) tube. In this system, then, the THMs present within the drinking water sample pervaporate through the silicone rubber membrane and are collected in the reagent water, while the HAAs remain in the drinking water sample and thus in the outer tube (i.e., they do not pervaporate through the membrane). The two water samples (drinking and reagent) are pumped through the CMS device using two separate peristaltic pumps, or possibly through a single pump having two channels for separate application. The resultant reagent stream (including pervaporated THMs) is called the acceptor stream, and is kept separate from the resultant drinking water stream (including non-pervaporated HAAs) called the donor stream. The two sample fractions are then collected in separate flasks, to which an appropriate amount of nicotinamide and base (such as, for example, sodium hydroxide) reagents are then added. The flasks are heated for an appropriate amount of time and cooled before the fluorescence intensity in the 450 nm region of each solution is measured (using a fluorescence spectrometer, fluorimeter, or handheld fluorimeter). The fluorescence intensity can be related back to the Total THMs or Total HAAs using a calibration curve or standard addition.

In terms of calibration techniques, one potential method is a calibration curve plotting known analyte concentrations against the signal, thus allowing for the determination of unknown concentrations of THMs and HAAs to be plotted on the known concentration curve. Unfortunately, the process of constructing such a calibration curve can prove time consuming and cumbersome, not to mention such curves may be compromised to a certain extent by unknown interfering species present within the subject standards. As such, development of such a curve, even with a semi-automated process (such as that provided within the inventive kit), is not practical, particularly when there is a need for quick turnaround in terms of drinking water sample analysis. To compensate for such potential impracticalities and possible matrix effects, a simpler approach has been followed that provides reliable standards.

A two-point standard addition (or “spike”) protocol has proven highly effective to provide the necessary and reliable curve for such a kit method. Standard addition itself is actually a system with two possible alternatives; one is highly similar to calibration curves and thus requires nearly the same amount of time and effort to develop. As such, for this inventive method, such a program is not desirable. The other, as noted above, is a spike method involving the analysis of an unknown concentration sample against the analysis of an unknown sample to which is added a known concentration of a similar compound (the “spike”). In such an instance, the concentration of the unknown in the original sample can be calculated from the equation:

$${[ X ]}_{i} = \frac{{[ S ]}_{f}}{( {I}_{s + x} / {I}_{x} ) - ( {V}_{0} / V )}$$

### Where:

Iₛ₊ₓ=Signal of the spiked sample

Iₓ=Signal of the original sample

[S]_{f}=Spike final concentration

V₀/V=Dilution factor

With a single THM and a single HAA (chloroform and trichloroacetic acid, for instance) used as the spikes for such measurements, the resultant drinking water samples can be analyzed with a certain degree of reliability as to the actual concentration of disinfectant by-products present within the tested samples through the utilization of the hand-held detector within the inventive kit. Such a “two point” standard addition technique better balances the quality of the desired result with the time available to carry out the technique under real-world condition and time constraints. However, either approach (graphical or two-point) may be employed if so desired by the operator, depending upon the potential need to balance the quality of the analytical measurement in use with limitations as to time.

As noted above, such a method permits quantification of both total trihalomethane and haloacetic acid species within the subject drinking water sample to determine the potential harmful levels of such suspect carcinogenic compounds therein. The method and the entire instrument may be operated by a single operator at any selected location along a drinking water supply line.

### BRIEF DESCRIPTION OF THE DRAWINGS

FIG. 1 depicts a representation of one potential embodiment of the overall kit.

FIG. 2 provides a schematic of the capillary membrane sampling module utilized within the inventive kit and method.

### DETAILED DESCRIPTION OF THE DRAWINGS AND PREFERRED EMBODIMENTS OF THE INVENTION

Without any intention to narrow the scope of the invention encompassed herein, hereinafter is provided some descriptions of potential embodiments that fall within the metes and bounds hereof.

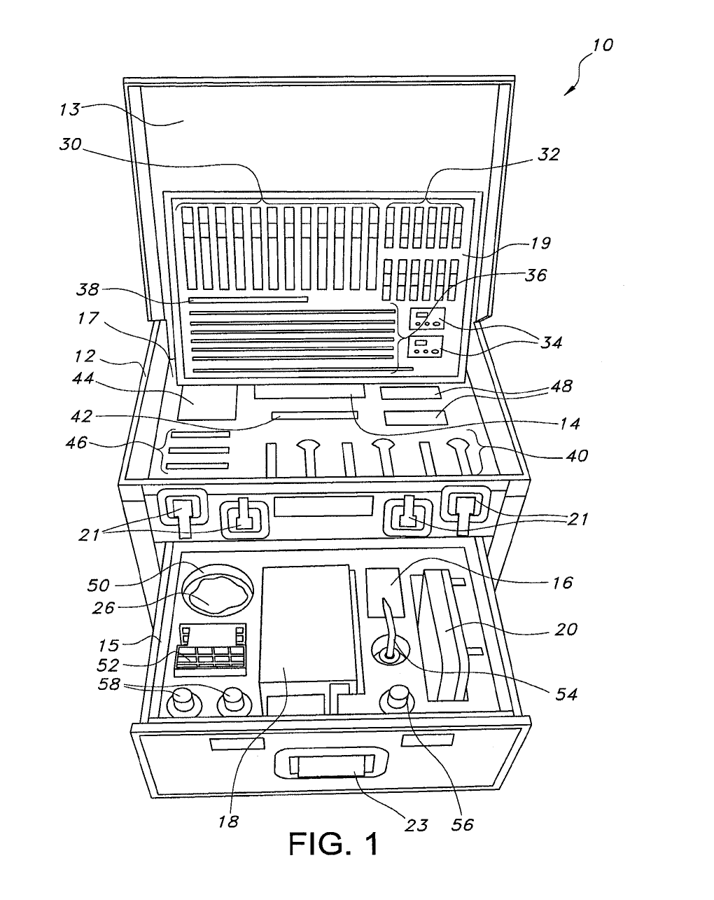

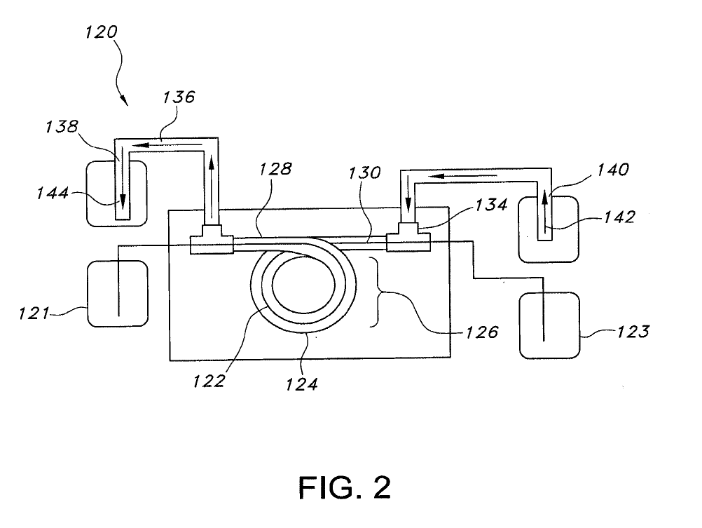

FIG. 1 shows a depiction of one potentially preferred embodiment of the inventive water testing kit 10, including a rugged carrying case 12 having a lid 13, a bottom drawer 15, a middle compartment 17, a folding top compartment 19, locking devices (to secure the case in a closed state) 21, and a carrying handle 23. Such a kit 10 and case 12 includes a hand-held fluorimeter 14, a capillary membrane sampling device 16, a peristaltic pump 18, a hot plate/stirrer 20, and an inflatable ice bath 26, as base components. Furthermore, in order to permit a user the capability of undertaking the necessary analytical steps involved within an all-in-one portable kit 10, the case also includes twelve small test tubes 30, twelve small cuvettes (also referred to as small test tubes) 32, two timing devices 34, pipettes of varying sizes 36 (and including a pipette bulb, if desired, not illustrated), a thermometer 38, six volumetric flasks 40, a graduated cylinder 42, extra tubing 44, three amber glass vials 46, two amber glass bottles 48, a 1000 mL beaker 50 (in which the inflatable ice bath 26 is stored), a test tube/cuvette holder 52, a wash bottle 54, a 250 mL bottle 56, and two 125 mL bottles 58. In practice, an operator would open the case 12 at a desired location and remove the hot plate 20 and ice bath 26. The hot plate 20 would be heated to 95° C. and kept at that temperature; ice would then be supplied for the ice bath 26 and kept at 0° degrees C., as well. The operator would then provide reagent and drinking sample water to the capillary membrane sampling device 16 through actuation of the peristaltic pump 14 attached to drinking water and reagent water supply lines (122, 124 of FIG. 2), and the collected samples therefrom would be present in two separate flasks 40 and then mixed with the nicotinamide reagent and a base (sodium hydroxide, preferably) (stored in the amber vials 46 or amber bottles 48. The resultant mixtures would then be heated on the hot plate 20 for a certain amount of time, then transferred to the ice bath 26 (whereupon the hot plate is turned off). After a designated period of time, and upon reaching a suitable temperature, samples of each flask 40 would then be transferred into individual cuvettes 32 and placed within the fluorimeter 14 for fluorescence detection and determination. From there, the operator may then register the fluorescent results and compare with standard curves to determine the amount of THM and HAA species present within the drinking water sample. The operator may then clean the used labware with the wash bottle 54 and place such kit components within the case 12 for further use. The case 12 should be suitably strong and rugged to protect the fragile components of the kit 10 during transport and storage. In terms of the kit 10, itself, the components are considered as modules constituting the entire portable analytical system. Thus, a capillary membrane sample module is present including the CMS device 16, the peristaltic pump 18, and at least two volumetric flasks 40. Such a module allows for separation of THM and HAA components from water samples and collection thereof for analysis thereafter. Also included is a nicotinamide fluorescence chemistry module including the necessary reagents for reaction for such analysis to occur (such as nicotinamide and a base, as noted above, in bottles 48), and vials 46 for mixing with water stream samples. A fluorescence detector module thus includes a hand-held fluorescence detector (such as a fluorimeter 14) and at least two cuvettes/test tubes 32 (or possibly 30, if desired), for introduction within the fluorescence detector 14. Lastly, an accessories module would include a hot plate 20 for a water bath (within a 1000 mL beaker 50, for instance), an ice bath 26, and a thermometer 38. The remaining components as shown within the potentially preferred kit 10 described herein would be considered part of the accessories module, at least. Such modules are described in greater detail, below.

In greater detail, FIG. 2 provides a view of the CMS (16 of FIG. 1, for instance). The overall CMS module 120 includes a 120 cm length of silicone rubber membrane tubing 122 placed inside Tefzel® tubing 124, with the resultant device 126 coiled. The two ends of the membrane 128, 130 were directed through two separate tee unions 132, 134 (Valco) and adapted to a 1/16″ to ⅛″ interior/exterior union 136 (Valco) using two stainless steel tubing sections 138, 140 (o.d. 0.5 mm, length 13 mm, Global FIA). The “sample in” 142 and “sample out” connections 144 consisted of 1/16″ FEP tubing adapted to fittings 146, 148 (Valco) using two 1/16″ to ⅛″ interior/exterior unions 150, 152 (Valco). Additional pieces of Tefzel® tubing were used to adapt these connections to the tees. Furthermore, 1/16″ fittings (Valco) were used to secure the FEP tubing to the unions (Valco). The CMS 120 was used to segregate THMs or HAAs species, as noted above. The dimensions provided here, as noted above, are not intended to be the sole measurements for such a purpose; various modifications in sizes and dimensions may be employed, if needed.

### Kit Description

As noted above, the kit itself is actually comprised of four different modules to provide four different actions during the drinking water testing procedure. In greater detail, then, the modules are describes thusly:

1) The Capillary Membrane Sampling Module primarily includes the CMS component (device) described in FIG. 2, above. This device is of relatively low cost and provides a highly effective manner of separating THMs and HAAs from drinking water samples, again, as described above. In previous analytical methods of drinking water measurements, automated analytical devices have been employed. In the current invention, however, the capillary membrane sampling device is utilized to provide the separation solely; subsequent to sequestration of the tested species, the operator then manually transfers the resultant samples to the other modules of the kit.

This CMS module also includes pumps (or a single pump) to deliver the drinking water and reagent water samples through the CMS device, and includes two flasks (or collection vessels, as an alternative description) for such a purpose. The acceptor stream of reagent water thus collects in the “HAA flask,” and the donor stream collects in the “THM flask.” These were then transferred manually to the next module.

2) The Nicotinamide Fluorescence Chemistry Module includes the nicotinamide and base reagents in order to provide the needed fluorescence to the HAA and THM samples. The two samples generated by the capillary membrane sampling module (the “THMs” flask and the “HAAs” flask) were subjected to reaction with 37.5% nicotinamide (37.5 mg) and base (sodium hydroxide, preferably)(7.5 M, 15 mg), mixed, and heated at 95° C. for 15 minutes. The samples were cooled using an ice bath (0° C.) and the fluorescence intensity of the solution in each flask were measured using the handheld fluorimeter of the Detection Module.

3) The Detection Module includes a battery-operated hand-held fluorimeter, as well as the necessary cuvettes into which fluoresced samples are introduced for placement and measurement within the fluorimeter. Calibration curves have been quantified and standardized (and thus would be part of the overall kit, as well) with regard to the fluorescent products of nicotinamide with THMs and HAAs in drinking water, using high-end fluorescence spectrometers or HPLC fluorescence detectors. These modes of detection provide the best results, but are costly and not portable (which thus led to the utilization of the present hand-held, battery-operated device of the inventive kit and method). From these curves, the operator may then input the readings from the fluorimeter for each species of THM and HAA to determine a reliable measurement of all such species present within the subject drinking water samples.

4) The Accessories Module of the inventive kit is basically the other components of the overall system that do not specifically provide actions, such as membrane sampling, fluorescing, or fluorescent detection. Thus, this module includes the necessary equipment and reagents to carry out the overall method. This includes labware (flasks, pipettes, vials, etc.), a hot plate, an ice bath, sample and reagent bottles, spare pump tubing, and a collection tray.

One potentially preferred, though not required and thus a non-limiting, outlay of an entire inventive kit would include the following components:

as equipment: a fluorescence detector, two cuvettes (or test tubes), a 2-channel pump with 0.64 mm ID tubing, 2-100 mL volumetric flasks, 2-250 mL volumetric flasks, a 120 cm Capillary Membrane Sampling device (CMS), a water bath with lid, an ice bath (both baths sufficiently large to hold two 250 mL volumetric simultaneously), a timing device, 4-65 mL vials, 2 Thermometers, and 4-43 mL vials;

as reagent and standard chemicals: 7.5 M Sodium hydroxide (Fisher), 37.5% Nicotinamide (Sigma Aldrich), 0.1560 M EDTA, standardized Chloroform (Sigma), and standardized Trichloroacetic Acid (Sigma Aldrich)(the reagent chemicals are pre-mixed to provide specific concentrations for introduction within the drinking water samples; for instance, 15 g Sodium Hydroxide was introduced into 50 mL volumetric flask and filled with reagent water to the specified line; 37.5 g of the Nicotinamide was introduced into a 100 mL volumetric flask and filled with reagent water to the specified line; 22.8101 g of EDTA was introduced into a 500 mL volumetric flask and filled with reagent water to the specified line)(the standardized ones were provided to allow the operator to properly calibrate the fluorimeter prior to undertaking measurements of drinking water samples). Additionally, the kit may include oxalic acid, as discussed below, for utilization within the overall method, as well.

Method of Use of the Kit

The overall procedure thus preferably follows the these sequential steps undertaken by the operator:

1) Turn on the hot plate and fill the water bath with enough water to cover the sample completely.

2) Flush the CMS device with reagent water for 15 minutes.

3) During the 15 minutes, prepare 7.5 M sodium hydroxide, 37.5% NCA, 1000 μg/L Trichloroacetic acid spike standard and 1000 μg/L Chloroform spike standard.

4) Perform dilution (particularly if the disinfectant by-product concentration in the water sample is not within the linear range of the kit) on the water sample accordingly, as needed in a 250 mL volumetric flask.

5) Check the pH of the water sample before sampling process to make sure the water sample is higher than pH 6.

6) Place the tubing in the sample flask and cover with parafilm.

7) Turn on the pump and set the timer for 3 minutes to fill CMS device with sample.

8) Stop the pump and place the ends coming out of the CMS in their respective collection 43 mL vials and cover each with their respective lids. As noted above, the liquid flowing through the silicone membrane will be collected in the acceptor vial and the liquid flowing over the silicone membrane will be collected in the donor vial.

9) Turn the pump on and set the timer for 43 minutes in order to collect the sample.

10) Stop the pump and remove the tubing from the collection vials and place the tubing into the waste beaker.

11) Add 0.0426 g of oxalic acid only to the donor stream sample vial and keep the sample in a refrigerator for 4 hour and 13 minutes. After about an hour, transfer the donor stream sample into a 65 mL sample vial and adjust the pH of the sample with 7.5 M sodium hydroxide to around pH 10 before adding 1 mL of 0.1560 M EDTA. Place the sample back in the refrigerator until the initial time duration noted above has ended. (This step is not followed for the acceptor stream vial.)

12) When a sample collected after the sampling process is ready to be analyzed, add 15 mL of respective sample collected to a 65 mL vial and followed by 1 mL of 7.5 M sodium hydroxide and 5 mL of 37.5% Nicotinamide.

13) Add a stir bar to each vial.

14) Make sure the water bath is around 100° C.

15) Place the first vial into the water bath and set the timer for 15 minutes.

16) Remove the first vial in a thermos and fill it up with ice and water.

17) When the sample in the first vial reaches 5° C., count 5 minutes after that point and then take the fluorescence reading.

18) Repeat steps 12) to 17) for the second vial.

19) Remove the tubing from the sample flask and place it back into the reagent water.

20) Turn the pump on and flush with reagent water for about 45 minutes.

21) To prepare a spike sample, the water sample is mixed with an amount of either standardized Chloroform or Trichloroacetic acid in a 100 mL volumetric flask until the final concentration of both THMs and HAAs are still within the linear range of the fluorimeter detection limits of the kit (10-100 μg/L).

22) Repeat steps 5) to 17) for the spike samples.

23) After sampling the last sample, detach the C-18 cartridge from CMS and flush it with about 6 mL of methanol then flush with reagent water at 1 mL/min for 45 minutes.

The fluorescence detection step is then performed as follows:
1) Clean the square plastic cuvettes with reagent water and then dry same until no moisture is present (preferably with a non-abrasive wipe).
2) Rinse the sample cuvette 2 times with sample before filling for analysis.
3) Press the “on/off” button to turn on the detector.
4) Place the cuvettes filled with sample in the cuvette holder and press the “read” button on the detector.
5) Record the fluorescence reading appears on the screen.
6) Do this for all samples.

### Fluorescent Chemical Considerations for Precise Measurements

As alluded to above, the fluorescent analytical method is based on the reaction of THMs and HAAs with NCA to form fluorescent products. It is believed that such a method is distinguishable from other procedures followed in past developments. To that end, the NCA reaction is not the same as that performed within a typical Fujiwara colorimetric reaction wherein nitrogenated polyenes are formed when dihalogenated or trihalogenated species react with pyridine. In that process, N-alkylated pyridines are formed when a polyhalogenated compound undergoes nucleophilic substitution by pyridine. Such a reaction is first order in pyridine and alkyl halide wherein the N-alkylated pyridine ring cleaves in alkaline solution to form an hydroxylated intermediate by attack at the pyridinium-2 position. Hydrolysis of this compound yields the salt of the amidine with an absorption maximum (λₘₐₓ) at 420 nm. If the pyridine concentration is high, an alkyl-pyridinium intermediate is formed. Hydrolysis of this compound yields a different amidine sodium salt (λₘₐₓ=530 nm). Each of these absorbing products then slowly decompose to form glutaconaldehyde (λₘₐₓ=370 nm).

Such a method is similar to that followed by Hach Chemical within its own fluorescent measurement kits; however, such a procedure does not permit any differentiation between the total amounts of THMs and HAAs within such analyzed drinking samples, not to mention there is no manner of determining concentrations of such species that are relevant to regulations set by the USEPA.

Apparently, such Fujiwara-based analyses are based on ultraviolet-visible (UV-Vis) methodologies that do not permit sufficiently low method detection limits (MDLs) for the concentrations needed for drinking water analysis. This is because the molar absorptivities of the resultant fluorescent products are simply not large enough for analysis at sub-μg/L concentration levels (as is typical within such drinking water measurement situations).

To the contrary, then, the utilization of NCA provides a different pyridine derivative having a formamide moiety meta to the N in the aromatic ring. The fluorescent products including such a reagent compound permits acceptable fluorescence as well as suitable sensitivities for low-level measurements of drinking water disinfectant by-products at the sub microgram levels needed for proper and reliable analysis. In the past, however, such NCA utilization has been limited to liquid chromatography-mass spectrometry detection to identify the products of the reaction between NCA and HAAs. In general, such a reaction is complex, but ultimately yields glutaconaldehyde derivatives, and thus is similar to Fujiwara chemistries, although, again, the level of detection permitted through NCA use specifically, is well below the detection limits accorded standard Fujiwara pyridines.

### Reliability and Measurement Modifications for Hand-Held Fluorescence Detection

The utilization of a portable fluorescence detector within the inventive kit, although of necessity for, again, the portability and accessibility of the overall method for employment by a human operator at remote locations, has also been rather difficult in practice to ensure reliable and consistent readings. In effect, the past utilization of non-portable instruments, such as, post column reaction ion chromatography, as one example, provided highly reliable results, but with the trade off of overall expense and single location placement (even though such a device could be placed at a remote distance from a water utility source and removed the need for a human operator). The necessity to overcome such a single location placement requirement, as well as the removal of a human operator, particularly with a portable system that could be utilized anywhere it could be transported along a drinking water supply line, led to the reduction of the fluorescence detector module to a portable (hand-held, as one example) type as described herein. For instance, an AQUAFLOR® device with analytical capabilities for measuring fluorescence as well as turbidity of liquids is one potentially preferred fluorimeter that meets such limitations. In such a device, the turbidity component may be exchanged with a second fluorescence detector, into which, as with the other, a cuvette with fluorescing liquid may be introduced and subject to proper measurements for levels of fluorescing compound present.

As alluded to above, such a device requires a certain degree of adjustment, particularly in the final measurement readings made thereby, to attune the overall result to that provided through more reliable means, such as, for instance, the ion chromatography prior methods employed for similar water species measurements. As such, it was important to assess the potential need for such modifications for such hand-held devices, in order to ensure the readings taken therewith would be acceptable from a USEPA guidelines perspective, as well as from an overall water treatment protocol standpoint in response thereto.

As such, preliminary studies were conducted with the overall inventive kit whereby standard concentrations of THMs and HAAs were first analyzed to generate plots of calibration curves for this hand-held device attentuation purpose. The resultant fluorescence intensity data from these standards was plotted as a function of either the THMs or HAAs concentration and the slope and y-intercept were determined using linear regression. The curves for both types of species were linear (with correlation coefficients greater than 0.99). An additional standard, not used in constructing the calibration curve was analyzed seven consecutive times and used to calculate the concentration of the check standard (for both total THMs and total HAAs); such a check provided a measure of the “experimental” concentration of total THMs or total HAAs to compare to the theoretical concentration (the concentration of total THMs and total HAAs in the check standard). Using these two numbers, it was possible to calculate three analytical parameters: the MDL (the lowest concentration distinguishable from noise), the mean % recovery (a measure of the accuracy of the overall measurements in terms of the proximity of the measured value to the true value of each sample's concentration), and the precision expressed as % relative standard deviation (a measure of the scatter or variation in the data). For total THMs, the MDL was 12 mg/L, with mean % recovery of 95% and % RSD of 5%. For total HAAs, the MDL was 10 mg/L, the mean % recovery was 102%, and the % RSD was 3%.

These standard measurements were then used to compare with actual working studies of the inventive kit used for field testing over several months. The resultant average MDL estimates for total THMs and total HAAs were 15.9 and 14.4 μg/L, respectively. The mean % recoveries working within a range of 4-5 of the MDL were 92.1 and 102.6% for total THMs and total HAAs, respectively, as well. The USEPA suggests that working with a range of 2.5 to 5 of the MDL, mean % recoveries of ±50% are acceptable. The measured disinfectant by-products via the inventive kit were % RSD estimates of 22.7 and 18.2% for total THMs and total HAAs, respectively, too. USEPA recommendations suggest that working with a range of 2.5 to 5 of the MDL, % RSD values may be ±30%. Therefore, the overall inventive kit results were well within range of “acceptable” for such purposes.

### Water Sample Analysis and Initial Measurement Limit Considerations

Beyond such initial considerations, however, is the overall measurement capability of the portable fluorescence detector to provide proper readings that a water utility may rely upon for proper treatment protocols to be undertaken in response to high disinfectant by-product levels. In order to assess such important issues, the inventive kit was compared with two reference methods in a side-by-side comparison for measuring total THMs and total HAAs in drinking water samples. Such water samples were taken from a Tennessee water utility in duplicate for this purpose with the comparison method being a gas chromatography reference procedure called on-line monitoring-purge and trap gas chromatography (OPTGC) as well as total HAAs measurements compared on their own between the inventive kit and post column reaction-ion chromatography (PCR-IC) as the reference. Both of these non-portable methods have been shown to compare favorably with USEPA Methods 502.2 and 552.3 in the past. In these situations and side-by-side comparisons, the reference methods are taken as “true values” due to the their consideration as a “higher order” methods of species detection. As a result, a bias between the kit and reference method(s) was estimated in the following equation (and manner):

Bias=“Experimental Method”−“True Value”

Thus, upon substitution of the results of the above-described methods, the equation becomes:

Bias=Inventive Kit Measurement−Reference Method Measurement

It was determined from these methods that the total THMs values using the inventive kit method predicted 16.5±0.6 μg/L while the OPTGC method predicted 21.5 μg/L. Thus, the average bias was −5.05±0.6 μg/L. As such, for total THMs it was understood that in the given system, the inventive kit will, on average, under-report (i.e., generate a slight negative bias) and such a bias can be utilized as a predictor of the concentration a reference method would expect in view of the inventive kit measurements. Such a predictor was considered generally sound since the nicotinamide chemistry tracks very well with total THMs and total HAAs, even when the bias was determined to be relatively large. For Total HAAs, then, the inventive kit measurements result were 66.6±2.4 μg/L and the PCR-IC reference method result was 14.2 μg/L. The bias was then calculated to be 52.5±2.4 μg/L. Thus for total HAAs, in this system, the inventive kit over-reported (i.e., generated a positive bias) the total HAAs by ˜52.5 μg/L with ˜4.6% error. Thus, as for the THM measurements, the inventive kit may then be attuned to predict the PCR-IC result (or any other reference method, for that matter) in relation to such a standard bias reading.

With fully automated (i.e., non-portable) instruments, the nicotinamide fluorescence methods behaved very well THMs and HAAs, and particularly in relation to the standard USEPA methods, noted above. Analyte tracking was evaluated more quantitatively, however, to further assure that the portable device of the inventive kit and method would provide reliable measurements, regardless of the need for bias compensation, as discussed above. To do so, the instantaneous concentration change for two adjacent samples were calculated at a particular point in time for two reference method (CMS-FIA and USEPA 502.2), as a basis for further comparison between the inventive portable method and such reference methods. At each point in time, the concentration at sampling time T1 and sampling time T2 (where T2 is greater than T1 and the two times are in consecutive order in the study), was calculated by subtracting the concentration of analyte at T1 from the concentration of analyte at T2. The two methods agree if both exhibit positive, negative or no change in concentration. By rule, the two methods would agree 33% of the time randomly; it was determined, though, that the CMS-FIA was in agreement with EPA 502.2 method for THM measurements within a chlorinated system 62.3% of the time. For HAA measurements within the same system, the CMS-FIA agreed 58.5% of the time with EPA 552.3. In a chloraminated system, the CMS-FIA agreed 60.8% of the time with EPA 502.2 for THM measurements and 62.7% of the time for HAA measurements.

Taken the bias measurements into account, and in view of these reference method comparisons, the inventive kit and method can thus properly predict the actual measurements within tested water samples within an error of about 11.9%. Such a level is acceptable for most water utilities seeking to optimize water treatment processes, particularly as such operators will be able to calibrate or “tune” such an inventive kit (and method) to predict the result that would be reported by selected reference methods such as USEPA 500 series methods for Total THMs (the compliance monitoring methods)²³ or Total HAAs through such a bias standard. Thus, such an inventive kit and method may reliably predict the concentrations of the two most commonly regulated drinking water disinfectant by-products as measured by higher order reference methods to a level of about ˜12% for total THMs and about 5% for total HAAs.

To further confirm these studies, the inventive kit and method was then utilized to further test water samples from three different locations (two additional Tennessee locations and one from Arkansas) to check for such reliability in bias measurements. The samples were collected in duplicate and run twice within one day with the THM concentration measurements made with both the inventive kit and OPT-GC and the calculated concentrations for total HAAs were compared between the inventive kit and those made via PCR-IC. The resultant inventive kit and method measurements reflect a slight negative bias over the three sample sites (averaging −10.0±1.8 μg/L, or about 18% relative error). Within a given sample site working at the MDL of the method, the inventive kit and method gave acceptable results (ranging from −3.1 to −35.4% RSD). Similarly, the results for Total HAAs showed an average bias of 29.5±8.6 μg/L (˜29% relative error). Both bias readings were acceptable by USEPA standards. Thus, the inventive kit and method, as noted previously, can be used to predict both the concentration of total THMs and total HAAs as reported by fully automated reference methods (OPT-GC and PCR-IC), respectively. Within a given sample site, the % RSD estimates range from very good (˜0.2%) to acceptable (˜30%), as well.

### Further Compensation Needs for Portable System Utilization

As discussed above, fully automated methods of compound detection, particularly fluorescence detection, is generally considered more reliable in terms of resultant measurements, particularly due to the capability of the analytical instrument to accord proper parameters and results for such a purpose. In terms of the utilization of nicotinamide as a fluorescing reagent, and the resultant reliance upon the believed-to-be-formed glutaconaldehyde fluorescing compound (without intending to rely upon any specific scientific theory for such a conclusion), it is understood that the intensity of the fluorescence exhibited by such a compound is proportional to the radiation which was absorbed by the species, thus providing a manner of measuring the initial amount of a starting compound within a tested sample. This is expressed in the following equation:

I=K(I₀−I_{T})

where:

I_{F}=intensity of the fluorescent light

I₀=intensity of the incident light

I_{T}=intensity of the transmitted light

K=a constant (function of quantum efficiency of fluorescence process)

The Beer-Lambert law may be expressed as:

I_{T}=KI₀10^{-abc}

Substitution of the second equation into the first yields:

I_{F}=KI₀(1−10^{-abc})

The exponential term in the equation can be expanded as a Taylor series giving:

$${I}_{F} = K {I}_{0} [ 2.3 a b c - \frac{{( 2.3 a b c )}^{2}}{2 ! } + \frac{{( 2.3 a b c )}^{3}}{ 3 !} + … ]$$

Traditionally, if abc<0.05, all the bracketed terms in the previous equation except the first term can be neglected and we may write:

I_{F}=K′I₀abc

Thus, the intensity of luminescence is proportional to the intensity of the incident beam, the absorptivity of the species, the pathlength of the cell, and the concentration of the fluorescent species within the limit that abc<0.05.

In relation to the presence of glutaconaldehyde, such a result has been reasoned to have an effect upon the portable device readings of the inventive kit and method. For example, the UV absorption spectrum of glutaconaldehyde is known to exhibit an a measurement of 58,884 M⁻¹ cm⁻¹. With a path length (b) of 1 cm, and a 1:1 stoichiometric conversion of THMs and HAAs species to glutaconaldehyde within the tested samples, then abc for chloroform ranges from 0.006 to 0.050 absorbance, which, in turn, corresponds to 12 to 101 mg/L. Using the same assumptions, abc for trichloroacetic acid ranges from 0.006 to 0.050 fluorescent intensity, which corresponds to 16 to 139 mg/L. Thus, calibration plots of such species would be expected to exhibit slight non-linearity beyond 101 mg/L for chloroform and 139 mg/L for trichloroacetic acid, potentially skewing the data and leaving the operator with potential problems in assessing the actual measurements made thereby. As a result, for the inventive kit and method, since a definitive path length within the fluorescence detector is followed (via the cuvette or test tube, for example, to be placed therein), caution must be exercised to ensure that the samples tested remain within the linear range when a standard addition calibration approach is undertaken. As noted above, for time sake and simplicity purposes, such a standard addition calibration method, such as a two-point procedure, will most likely be followed.

One manner of compensating for this issue is the generation of calibration curves. As noted above, however, this process is not conducive to such a remote-location, quick monitoring process. Thus, the utilization of a “spike” procedure requires care by the operator to ensure that the analyte concentration present within the samples does not exceed the above-noted limits of linear assurances. Failure to stay within the linear range can generate unacceptable error in the determination of the concentration. Thus, with this chemistry it is particularly important to know the linear range to a great deal of certainty and to operate within this range. Thus, to stay within the linear range, the operator must stay below 101 mg/L for total THMs and 139 mg/L for total HAAs. One manner of assurance is to dilute a sample accordingly before the sampling process, then spike the sample with total THMs and total HAAs up to the high end of their respective linear ranges. This keeps the sample and the spike results within the linear range as required by standard addition. Most likely, any water utilities that are interested in using such an inventive kit would be experience trouble meeting the USEPA regulations in terms of the presence of THMs and HAAs initially. In such a scenario, the total THMs would be most likely measure near 80 mg/L and total HAAs near 60 mg/L. An appropriate dilution from such a level (say, four to one, for example), would still render the resultant species levels near their MDL values. The resultant diluted samples could then be spiked back up to the high end of the linear range and the spike could be used in conjunction with the sample to determine the analyte concentration. Thus, in real-time and real-life practice, a water utility could strategically plan any necessary dilutions accordingly, based upon historic THM and HAA concentration levels. Using such historical concentration measurements as suitable references, the operator can then undertake a proper dilution, if needed, to ensure the concentration of analyte in the sample and the spiked sample is within the linear range of the inventive portable fluorescence detector kit and method. Such a necessary compensation scheme is solely needed in terms of the issues involved with portable detectors; as noted above, the fully automated prior methods (reference methods) do not require such modifications.

Thus, the overall invention described herein provides a relatively simple, yet reliable, field portable kit for routine monitoring studies of the total trihalomethanes and haloacetic acids (the two most regulated water system disinfectant by-products). The kit exhibits good minimum detection limits, accuracy and precision for such a purpose, all within the guidelines set by the USEPA. The kit also exhibits a linear range of reliable measurements from ˜10 to ˜100 μg/L for total THMs and/or HAAs, thus permitting, again, an effective manner of testing for such potentially low levels of such species.

As well, the practice of two-point standard addition calibration, in a guarded manner, at least, also greatly simplifies the manual labor for using the kit in terms of ultimately assessing the measurements of fluorescent compound detection readings as markers of concentrations of such THM and HAA species. As noted above, the standard deviation of the bias for both total THM and HAA within a sample site ranged from 0.1 μg/L to 8.2 μg/L while the % Relative Standard Deviation (% RSD) ranged from 0.2% to −35%. The standard deviation of the average bias across the 3 sample sites for total number of trihalomethane species is 1.8 μg/L and for total haloacetic acid species is 8.6 μg/L, too. Such results indicate consistent bias within a sample site, thus permitting reliable employment of such a kit and related test method to predict the concentration of USEPA compliance monitoring methods and accord a pertinent water utility the capability of economically monitoring water samples for disinfectant by-products in a real-time manner to permit water utility optimization of water treatment practices quickly, reliably, and cost-effectively.

The preceding examples and discussion are set forth to illustrate the principles of the invention, and specific embodiments of operation of the invention. The examples are not intended to limit the scope of the method. Additional embodiments and advantages within the scope of the claimed invention will be apparent to one of ordinary skill in the art.

What we claim is:

1. A portable kit for analyzing drinking water samples comprising:
a) a capillary membrane sampling module comprising:
a capillary membrane sampling device, a 2-channel pump, and at least two volumetric flasks;
b) a nicotinamide fluorescence chemistry module comprising:
reagent supplies of nicotinamide and a base, and at least two vials for mixing said nicotinamide reagent and said base reagent with at least two different water stream samples;
c) a fluorescence detector module comprising:
a hand-held fluorescence detector and at least two cuvettes that may be introduced within said fluorescence detector; and
d) an accessories module comprising:
a covered water bath that may be heated to at least 80° C. and an ice bath, wherein both said baths can hold both volumetric flasks within said capillary membrane sampling module simultaneously, and a thermometer to monitor the temperature of both baths;
wherein all of said modules are simultaneously portable and carried within a single properly configured and compartmented protective enclosure.

2. A method for analyzing drinking water samples through the utilization of a portable analytical device, comprising the steps of:
a) providing at least one stream of drinking water that has been disinfected with chlorinated or chloraminated materials;
b) providing a kit including four modules for analysis of said stream of drinking water in step “a”, said modules including i) a capillary membrane sampling module, ii) a nicotinamide fluorescence chemistry module, iii) a fluorescence detector module, and iv) an accessories module, wherein said modules may be properly operated by a single person throughout each procedure, and wherein said capillary membrane sampling module includes a capillary membrane sampling component including a tube-within-a-tube construction to allow two different streams of liquid to pass through said component simultaneously;
c) introducing said at least one drinking water stream into said capillary membrane sampling component through one tube and a reagent water stream through the other tube within said component such that device such that all volatile trihalomethane compounds present within said drinking water stream separates from said stream within said capillary membrane sampling device into a stream of reagent water, and wherein any haloacetic acid compounds will remain within said at least one stream of drinking water;
d) transporting both of said trihalomethane-containing stream of reagent water and said drinking water haloacetic acid-containing stream to separate reservoirs, wherein samples from each reservoir are removed therefrom and kept separate;
e) mixing said separate samples with a nicotinamide fluorescing compound within the nicotinamide fluorescing chemistry module;
f) optionally heating said nicotinamide-mixed separate samples to a temperature to effectuate reaction therewith between said separated trihalomethane compounds and said haloacetic acid compounds within different mixing vessels;
g) optionally cooling each resultant fluoresced separate sample thereof to a temperature that permits handling and introduction within a cuvette for placement within a fluorimeter, wherein said heating and cooling steps are undertaken within the accessories module;
h) introducing said resultant separate samples into separate cuvettes;
i) placing said separate cuvettes into a fluorescing detection instrument within the fluorescing detector module; and
j) operating said fluorescing detection instrument to determine the total amount trihalomethane and haloacetic acid species within each resultant separate sample through fluorescence detection.
